(19) United States
(12) Patent Application Publication
(10) Pub. No.: US 2023/0181510 A1
(43) Pub. Date: Jun. 15, 2023

(54) COMPOSITION AND METHODS OF RETINOIC ACID

(71) Applicant: Zhaoyang Li, San Jose, CA (US)

(72) Inventor: Zhaoyang Li, San Jose, CA (US)

(21) Appl. No.: 17/926,388

(22) PCT Filed: May 21, 2021
(86) PCT No.: PCT/US21/33726

§ 371 Date: Nov. 18, 2022

Related U.S. Application Data
(60) Provisional application No. 63/029,479, filed on May 24, 2020.

(51) Int. Cl. A61K 31/203; A61K 47/24; A61K 9/00; A61K 9/107; A61K 47/44; A61P 19/02; A61P 29/00

(52) U.S. Cl. CPC A61K 31/203; A61K 47/24; A61K 9/0014; A61K 9/107; A61K 47/44; A61P 19/02; A61P 29/00

(57) ABSTRACT

The present invention provides a composition for relief or management of a pain associated with a disorder, e.g., joint or skeletal condition, the composition comprises n effective amount of retinoic acid. Methods of making and using the same are also disclosed.

### CROSS-REFERENCE TO RELATED APPLICATIONS

This application claims the benefit to U.S. provisional application No. 63/029,479, filed May 24, 2020, the teaching of which is incorporated herein by reference in its entirety.

### FIELD OF THE INVENTION

The present invention relates to a composition of retinoic acid for a joint disorder or a skeletal disorder.

### BACKGROUND OF THE INVENTION

Acute and chronic body pain is common. Chromic pain associated with aging or body injury is becoming a social issue as use of narcotics, a prescribed item for pain management, is often abused, leading to many deaths. Abuse of narcotics creates impressing needs for alternatives for pain relief or pain management.

On the other hand, osteoarthritis (OA) is a disease where articular cartilages are deformed mostly by aging, causing pains and movement disorders. Normal articular cartilages are kept elastic by repetitive metabolism, but they become less elastic with age and are gradually worn out. As a result, bones which constitute a joint are in direct contact with each other; liga-ments and joint capsules are loosened, tensioned, and/or com-pressed; and these cause pain. As it progresses, eventually, rubbing of bones generates spinal spurs on bones, causing more pain. The main cause of OA is aging. As the society continues the course toward an aged society, a rapid increase in the number of OA patients is predicted.

For OA treatment, physical therapies and drug therapies by drug administration/injection are employed. If there is no symptomatic improvement, operations are performed. One of the drug therapies for OA is a direct injection of hyaluronic acid, a protective agent for articular cartilage, into the affected part. The intra-articular injection of hyaluronic acid can prevent rubbing of bones to remove pain. The improvement of j oints by hyaluronic acid injection is an excellent therapeutic method which can restore the patient's QOL. However, in order to attain the therapeutic effect of this method, the hyaluronic acid injection is normally required once a week for about five weeks. Moreover, the attained analgesic effect is not permanent, and treatment needs to be recommenced after a certain period of time, which increases the therapeutic burden on the patient.

Therefore, there is a continuing need for new compositions and methods for pain or skeletal conditions.

The embodiments provided below address the above identified issues and needs.

### SUMMARY OF THE INVENTION

In one aspect of the present invention, it is provided a composition for relief or management of a pain associated with a disorder in a subject in need thereof, the composition comprises a therapeutically effective amount of retinoic acid dissolved or dispersed in a carrier, and application of the composition to a site having the pain or disorder causes a recognizable reduction of pain in the subject.

In some embodiments of the invention composition, optionally in combination with any of the various embodiments disclosed herein, the disorder is of an organ, muscle, joint, or a skeletal structure.

In some embodiments of the invention composition, optionally in combination with any of the various embodiments disclosed herein, the disorder is of an acute condition or a chronic condition.

In some embodiments of the invention composition, optionally in combination with any of the various embodiments disclosed herein, the retinoic acid comprising 0.01% to 2% of the total weight of the composition.

In some embodiments of the invention composition, optionally in combination with any of the various embodiments disclosed herein, the composition further comprises a bone-targeting molecule for directing the retinoic acid to a skeletal tissue.

In some embodiments of the invention composition, optionally in combination with any of the various embodiments disclosed herein, the bone-targeting molecule is a bisphosphonate.

In some embodiments of the invention composition, optionally in combination with any of the various embodiments disclosed herein, the carrier is a non-aqueous carrier.

In some embodiments of the invention composition, optionally in combination with any of the various embodiments disclosed herein, the bone-targeting molecule is one of

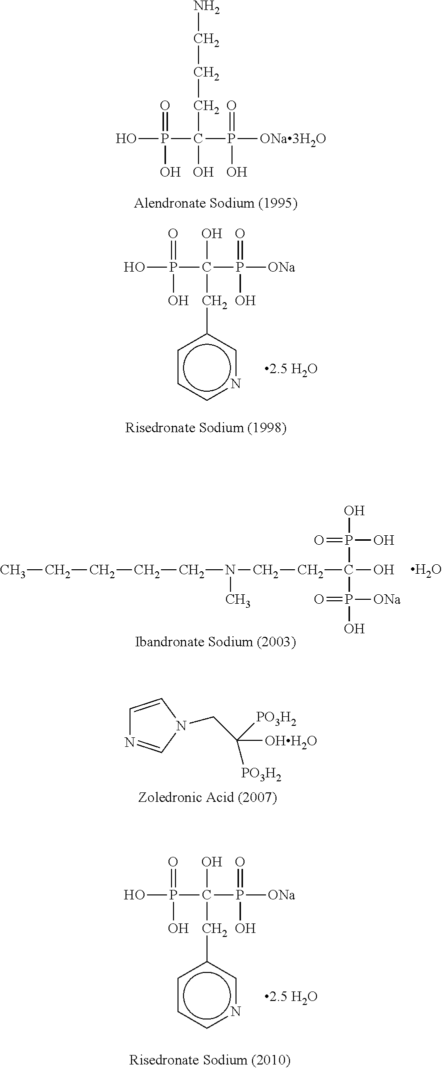

In some embodiments of the invention composition, optionally in combination with any of the various embodiments disclosed herein, the composition is in a formulation for local delivery or systemic delivery.

In some embodiments of the invention composition, optionally in combination with any of the various embodiments disclosed herein, the formulation for local delivery is a topical formulation.

In some embodiments of the invention composition, optionally in combination with any of the various embodiments disclosed herein, the formulation is an injection formulation.

In some embodiments of the invention composition, optionally in combination with any of the various embodiments disclosed herein, the formulation comprises a hydrogel or an emulsion.

In some embodiments of the invention composition, optionally in combination with any of the various embodiments disclosed herein, the formulation comprises hyaluronic acid, provitamin B5, anti-itching agent, and/or vitamin C.

In another aspect of the present invention, it is provided a method for relief or management of a pain associated with a disorder, comprising administering to a subject in need thereof a composition that comprises a therapeutically effective amount of retinoic acid dissolved or dispersed in a carrier to a site having the pain or disorder, causing a recognizable reduction of pain in the subject.

In some embodiments of the invention method, optionally in combination with any of the various embodiments disclosed herein, the disorder is of an organ, muscle, joint, or a skeletal structure.

In some embodiments of the invention method, optionally in combination with any of the various embodiments disclosed herein, the disorder is of an acute condition or a chronic condition.

In some embodiments of the invention method, optionally in combination with any of the various embodiments disclosed herein, the retinoic acid comprising 0.01% to 2% of the total weight of the composition.

In some embodiments of the invention method, optionally in combination with any of the various embodiments disclosed herein, the composition further comprises a bone-targeting molecule for directing the retinoic acid to a skeletal tissue.

In some embodiments of the invention method, optionally in combination with any of the various embodiments disclosed herein, the bone-targeting molecule is a bisphosphonate.

In some embodiments of the invention method, optionally in combination with any of the various embodiments disclosed herein, the carrier is a non-aqueous carrier.

In some embodiments of the invention method, optionally in combination with any of the various embodiments disclosed herein, the bone-targeting molecule is one of

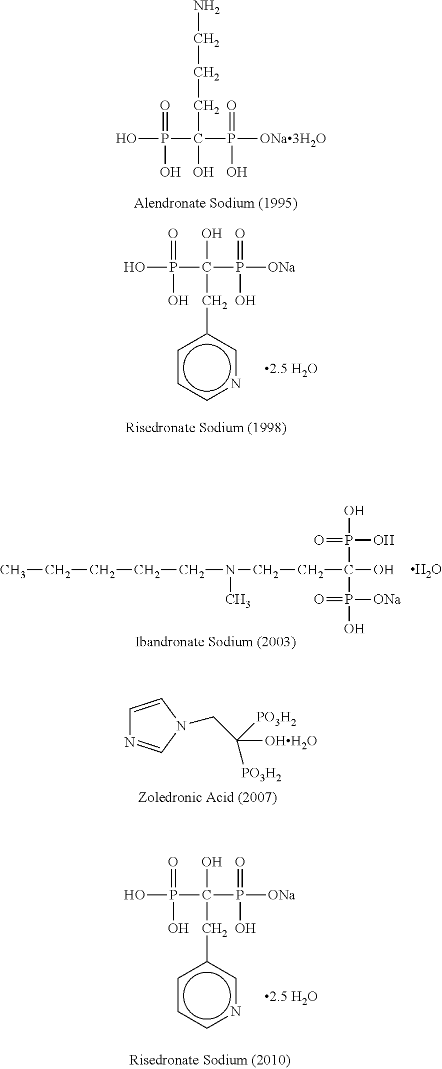

In some embodiments of the invention method, optionally in combination with any of the various embodiments disclosed herein, the composition is in a formulation for local delivery or systemic delivery.

In some embodiments of the invention method, optionally in combination with any of the various embodiments disclosed herein, the formulation for local delivery is a topical formulation.

In some embodiments of the invention method, optionally in combination with any of the various embodiments disclosed herein, the formulation is an injection formulation.

In some embodiments of the invention method, optionally in combination with any of the various embodiments disclosed herein, the formulation comprises a hydrogel or an emulsion.

In some embodiments of the invention method, optionally in combination with any of the various embodiments disclosed herein, the formulation comprises hyaluronic acid, provitamin B5, anti-itching agent, and/or vitamin C.

In some embodiments of the invention method, optionally in combination with any of the various embodiments disclosed herein, the subject is a patient, a pet or horse.

In a further aspect of the present invention, it is provided a method of using a composition for relief or management of a pain associated with a disorder in a subject in need thereof, the composition comprising a therapeutically effective amount of retinoic acid dissolved or dispersed in a carrier, wherein application of the composition to a site having the pain or disorder causes a recognizable reduction of pain in the subject.

In some embodiments of the invention method, optionally in combination with any of the various embodiments disclosed herein, the disorder is of an organ, muscle, joint, or a skeletal structure.

In some embodiments of the invention method, optionally in combination with any of the various embodiments disclosed herein, the disorder is of an acute condition or a chronic condition.

In some embodiments of the invention method, optionally in combination with any of the various embodiments disclosed herein, the retinoic acid comprising 0.01% to 2% of the total weight of the composition.

In some embodiments of the invention method, optionally in combination with any of the various embodiments disclosed herein, the composition further comprises a bone-targeting molecule for directing the retinoic acid to a skeletal tissue.

In some embodiments of the invention method, optionally in combination with any of the various embodiments disclosed herein, the bone-targeting molecule is a bisphosphonate.

In some embodiments of the invention method, optionally in combination with any of the various embodiments disclosed herein, the carrier is a non-aqueous carrier.

In some embodiments of the invention method, optionally in combination with any of the various embodiments disclosed herein, the bone-targeting molecule is one of

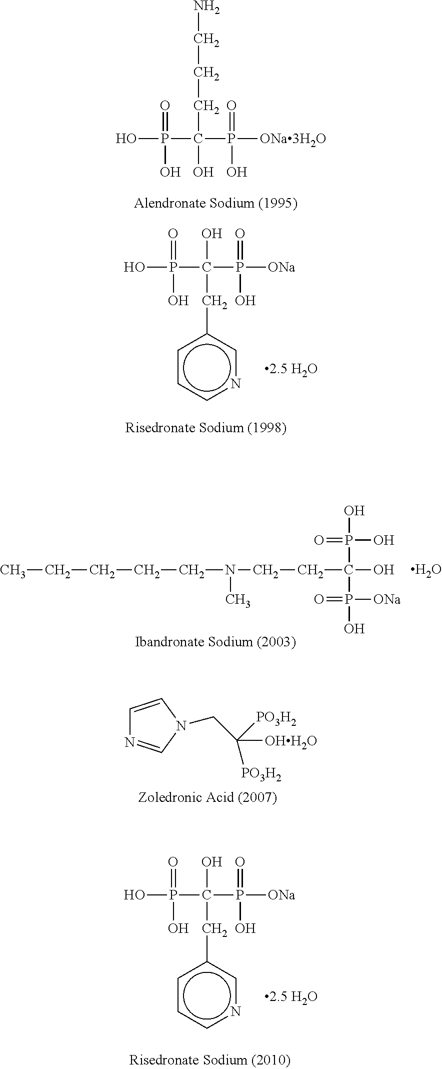

In some embodiments of the invention method, optionally in combination with any of the various embodiments disclosed herein, the composition is in a formulation for local delivery or systemic delivery.

In some embodiments of the invention method, optionally in combination with any of the various embodiments disclosed herein, the formulation for local delivery is a topical formulation.

In some embodiments of the invention method, optionally in combination with any of the various embodiments disclosed herein, the formulation is an injection formulation.

In some embodiments of the invention method, optionally in combination with any of the various embodiments disclosed herein, the formulation comprises a hydrogel or an emulsion.

In some embodiments of the invention method, optionally in combination with any of the various embodiments disclosed herein, the formulation comprises hyaluronic acid, provitamin B5, anti-itching agent, and/or vitamin C.

In some embodiments of the invention method, optionally in combination with any of the various embodiments disclosed herein, the subject is a patient, a pet or horse.

In a further aspect of the present invention, it is provided a method of fabricating a composition for relief or management of a pain associated with a disorder, comprising providing a therapeutically effective amount of retinoic acid and a carrier, and dissolving or dispersing the therapeutically effective amount of retinoic acid in the carrier to form the composition.

In some embodiments of the invention method, optionally in combination with any of the various embodiments disclosed herein, the disorder is of an organ, muscle, joint, or a skeletal structure.

In some embodiments of the invention method, optionally in combination with any of the various embodiments disclosed herein, the disorder is of an acute condition or a chronic condition.

In some embodiments of the invention method, optionally in combination with any of the various embodiments disclosed herein, the retinoic acid comprising 0.01% to 2% of the total weight of the composition.

In some embodiments of the invention method, optionally in combination with any of the various embodiments disclosed herein, the composition further comprises a bone-targeting molecule for directing the retinoic acid to a skeletal tissue.

In some embodiments of the invention method, optionally in combination with any of the various embodiments disclosed herein, the bone-targeting molecule is a bisphosphonate.

In some embodiments of the invention method, optionally in combination with any of the various embodiments disclosed herein, the carrier is a non-aqueous carrier.

In some embodiments of the invention method, optionally in combination with any of the various embodiments disclosed herein, the bone-targeting molecule is one of

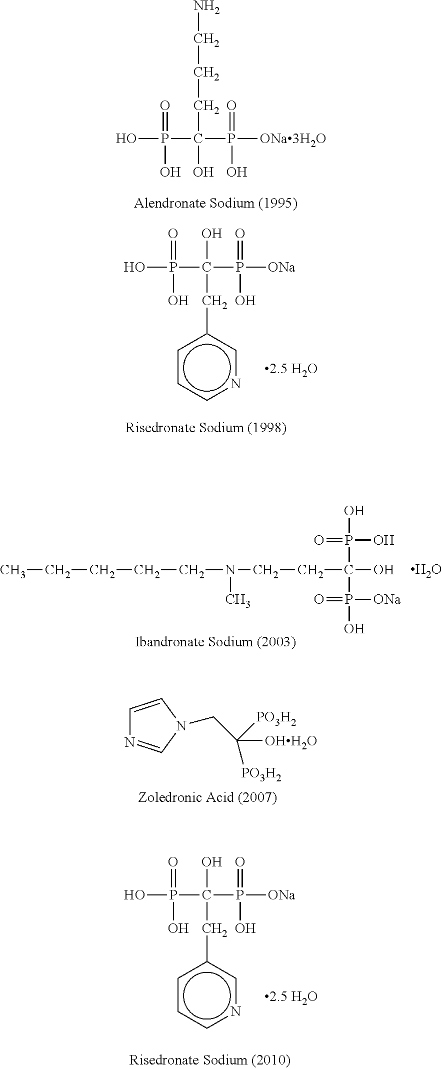

In some embodiments of the invention method, optionally in combination with any of the various embodiments disclosed herein, the composition is in a formulation for local delivery or systemic delivery.

In some embodiments of the invention method, optionally in combination with any of the various embodiments disclosed herein, the formulation for local delivery is a topical formulation.

In some embodiments of the invention method, optionally in combination with any of the various embodiments disclosed herein, the formulation is an injection formulation.

In some embodiments of the invention method, optionally in combination with any of the various embodiments disclosed herein, the formulation comprises a hydrogel or an emulsion.

In some embodiments of the invention method, optionally in combination with any of the various embodiments disclosed herein, the formulation comprises hyaluronic acid, provitamin B5, anti-itching agent, and/or vitamin C.

In some embodiments of the invention method, optionally in combination with any of the various embodiments disclosed herein, the subject is a patient, a pet, or a horse.

### DETAILED DESCRIPTION OF THE INVENTION

### Definitions

As used herein, all percentages are by weight (wt %) of the total composition.

As used herein, the term “therapeutically effective” shall mean a recognizable reduction of pain as measured according to a pain scale rating system commonly used by a medical practitioner in the field of pain medicine. See T. Bendinger, Measurement in Pain Medicine, in BJA Education, Volume 16, Issue 9, September 2016, Pages 310-315.

All numeric ranges are inclusive of narrower ranges; delineated upper and lower range limits are interchangeable to create further ranges not explicitly delineated. All ranges and values disclosed herein are inclusive and combinable. For examples, any value or point described herein that falls within a range described herein can serve as a minimum or maximum value to derive a sub-range, etc.

As used herein, the term “retinoic acid” refers to vitamin A or a derivative thereof:

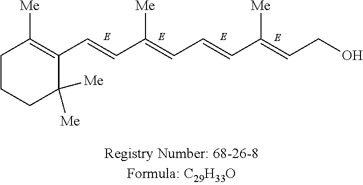 [SMILES: CC1=C(/C=C/C(C)=C/C=C/C(C)=C/CO)C(C)(C)CCC1]

Vitamin A has a number of isomers and derivatives, which are referred to as retinoid. A preferred retinoid is retinoic acid, which has a number of isomers. In some embodiments, a preferred retinoic acid is all-trans retinoic acid. Trans isomers are different from cis isomers as they have different structures and thus pharmacological effects on a subject receiving the retinoic acid.

Retinoic acid is a naturally occurring compound. This compound is widely used in skincare products for its antiaging effects, e.g., wrinkle reduction (see, e.g., Li et al., Topical stabilized retinol treatment induces the expression of HAS genes and HA production in human skin in vitro and in vivo in Arch Dermatol Res. 2017 May; 309(4):275-283).

Topical stabilized retinoic acid treatment is shown to induce the expression of hyaluronic acid (HA) synthase (HAS) genes and HA production in human skin in vitro and in vivo (Id.) and stimulates collagen regeneration (see, e.g., Weinstein G D, Nigra T P, Pochi P E, et al. Topical tretinoin for treatment of photodamaged skin. Arch Dermatol., 1991; 127:659-65).

As used herein, the term “mega dose vitamin C” refers to a daily dose of vitamin C at least 3 grams when taken orally. In some embodiments, the term refers to a daily dose of vitamin C at least 10 grams when taken orally. In some further embodiments, the term refers to a daily dose of vitamin C at least 30 or 50 grams when taken orally or injection (e.g., percutaneous injection, local injection, or IV). Vitamin C has been shown to promote collagen synthesis (see, e.g., McGartland C P, Robson P J, et al. Fruit and Vegetable Consumption and Bone Mineral Density: The Northern Ireland Young Hearts Project. Am J ClinNutr, 2004; 80(4): 1019-1023).

As used herein, the term “hydrogel” is defined as a three-dimensional network of natural or synthetic polymers having the ability to absorb a large amount of water or biological fluids. The polymers must be compatible. Some examples such polymers are hyaluronic acid, polyacrylate (e.g., carbomer 940), polyvinylpyrrolidone (PVP). Other examples of polymers can include the ones described in more detail below.

As used herein, the term “disorder” refers to a disorder that elicits pain and can be of an a condition of organ, muscle, joint or a skeletal structure, e.g., muscle pain, pain of arthritis such as osteoarthritis and/or rheumatoid arthritis that involves inflammation or pain. In some embodiments, the term also refers to lower back pain or an acute or chronical muscle pain. In some embodiments, the term disorder refers to any pain in a joint or organ.

Osteoarthritis occurs when the protective cartilage that cushions the ends of your bones wears down over time. Osteoarthritis symptoms often develop slowly and worsen over time. Signs and symptoms of osteoarthritis include:

Pain. Affected joints might hurt during or after movement.
Stiffness. Joint stiffness might be most noticeable upon awakening or after being inactive.
Tenderness. Your joint might feel tender when you apply light pressure to or near it.
Loss of flexibility. You might not be able to move your joint through its full range of motion.
Grating sensation. You might feel a grating sensation when you use the joint, and you might hear popping or crackling.
Bone spurs. These extra bits of bone, which feel like hard lumps, can form around the affected joint.
Swelling. This might be caused by soft tissue inflammation around the joint.

Another skeletal condition is rheumatoid arthritis (RHA). RHA occurs when your immune system mistakenly attacks healthy joints. RHA symptoms may include pain, swelling, stiffness, and loss of physical function in areas, such as your hands, wrists, shoulders, knees, and feet. Over time, these symptoms can lead to irreversible joint damage.

RHA may result from a combination of factors, including genetic predisposition, environmental influences, and your immune system, even though the exact cause is unknown.

Therefore, as used herein, in some embodiments where a condition is associated with osteoarthritis or RHA, the term “therapeutically effective” refers to a composition disclosed herein being effective to treat or alleviate or reduce any of the symptoms of a skeletal condition, e.g., osteoarthritis or RHA. In this context, the term “effective” shall exclude reduction of pain by a pain killer that blocks the pain signal transduction between a site of joint disorder or skeletal disorder and nerves of the central or peripheral nervous systems. An indication of such distinction is that pain reduction by a pain killer that blocks the pain signal transduction between a site of joint disorder or skeletal disorder and nerves of the central or peripheral nervous systems would reoccur once the pain killer is cleared off the body of a patient taking the pain killer. Examples of such pain killer can be an opioid, cannabidiol or a non-steroid anti-inflammatory drug (NSAID) such as aspirin or ibuprofen. In other words, “effectiveness” or “therapeutically effectiveness” of the inventive composition or method is indicated by a gradual fading or attenuation of pain over the course of treatment using the method or composition of invention, and upon termination of use of the invention composition, the pain would not return or would not return to a level comparable to the pain that a patient had prior to any use of the invention composition. In other words, using of the invention composition would cause a recognizable reduction of pain in the patient on the site of condition of a joint or skeletal structure, and such reduction of pain will not disappear after a course of treatment when the patient stops using the invention composition.

### Mechanisms of RA Action

While not bound by any specific mode of action, it is hypothesized that the combination of biological functions of RA add up to the overall outcome of RA on pain and skeletal and/or muscle pain. For example, it is reported that RA acts as an anti-inflammatory agent so as to reduce or minimize pain (see, Hiroshi Keino, et al., Anti-inflammatory effect of retinoic acid on experimental autoimmune uveoretinitis in Br J Ophthalmol 2010 June; 94(6):802-7; see also Schimidt and Gans, Tretinoin: A Review of Its Anti-inflammatory Properties in the Treatment of Acne in J Clin Aesthet Dermatol. 2011 November; 4(11): 22-29). RA is also reported to act as an immune modulator, see, e.g., Martje Erkelens and Reina Mebius, Retinoic Acid and Immune Homeostasis: A Balancing Act in Trends in Immunology, vol. 38 (3), pp 168-180, Mar. 1, 2017 (Review). As previously noted, RA also stimulates hyaluronic production and collagen production (see, Li, supra, see also Schwartz, et al., Topical All-Trans Retinoic Acid Stimulates Collagen Synthesis In Vivo in J. Investigative Dermatology, Volume 96, Issue 6, June 1990, Pages 975-978).

### Bone-Targeting Molecule

As used herein, the term bone-targeting molecule refers to a group of compounds capable of effecting bone- or cartilage-specific targeting. A group of such bone-targeting molecules are the bisphosphonates (BP). The BP can guide retinoic acid bonding to hydroxyapatite (HA) in bone tissue as BP preferentially binds to HA. The binding between them is reversible, and the BP-retinoic acid will slowly dissociate from HA when its concentration in extracellular fluid going down, which lead to a sustained release followed by a long-term biological effect to stimulate bone regeneration.

The HA in bone will work as a reservoir of BP-retinoic acid (also referred to as “BP-retinoic acid conjugate”), stimulating HA synthesis and collagen synthesis so as to increase HA concentration. As BP works to reduce bone resorption, the BP-retinoic acid conjugate would increase bone density more than the use of either one alone (Otherwise, if the dose of BP is below the minimum effective dose, the BP will only work as a targeting molecule).

Biphosphonate Approved by FDA

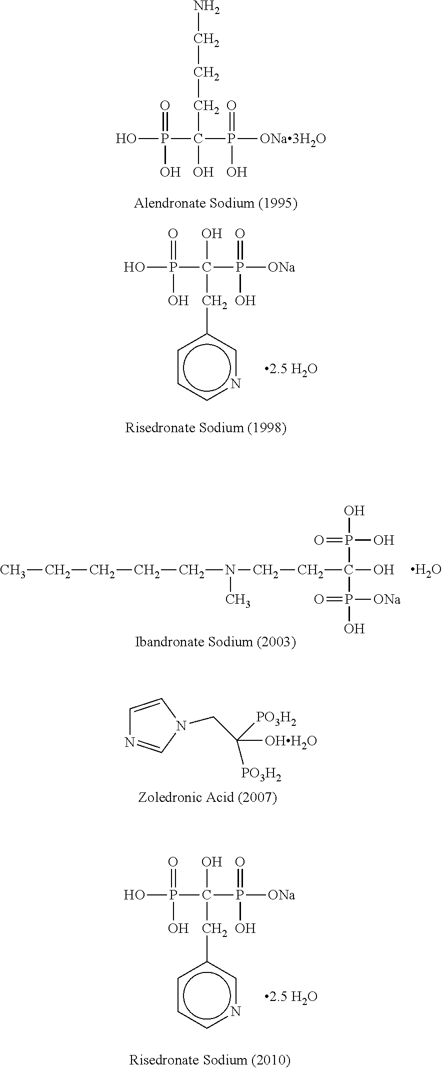

The BP-retinoic acid conjugate can be readily generated using well-documented chemistry. For example, a polyvalent metal salt that is biocompatible such a salt of Ca′, Al⁺³, Mg⁺², Ba⁺², Fe⁺³, Zn⁺², Ti⁺², Ti⁺⁴, Co⁺², Cr⁺², Cr⁺³, Zr⁺³, Cu⁺², Ni⁺², Ni⁺³, Ga⁺³ or a lanthanide ion can be used to conjugate BP and retinoic acid (e.g., retinoic acid forming a salt of the polyvalent metal along with the BP), forming the BP-retinoic acid conjugate. In some embodiments, a polymer carrying multiple positive charges can be used to conjugate BP and retinoic acid, forming the BP-retinoic acid conjugate. In some embodiments, retinoic acid can attach to BP via forming a bonding with the hydroxyl group(s) on BP so as to form a BP-retinoic acid conjugate.

### Composition

In one aspect of the present invention, it is provided a composition for relief or management of a pain associated with a disorder in a subject in need thereof, the composition comprises a therapeutically effective amount of retinoic acid dissolved or dispersed in a carrier, and application of the composition to a site having the pain or disorder causes a recognizable reduction of pain in the subject.

In some embodiments of the invention composition, optionally in combination with any of the various embodiments disclosed herein, the disorder is of an organ, muscle, joint, or a skeletal structure.

In some embodiments of the invention composition, optionally in combination with any of the various embodiments disclosed herein, the disorder is of an acute condition or a chronic condition.

In some embodiments of the invention composition, optionally in combination with any of the various embodiments disclosed herein, the retinoic acid comprising 0.01% to 2% of the total weight of the composition.

In some embodiments of the invention composition, optionally in combination with any of the various embodiments disclosed herein, the composition further comprises a bone-targeting molecule for directing the retinoic acid to a skeletal tissue.

In some embodiments of the invention composition, optionally in combination with any of the various embodiments disclosed herein, the bone-targeting molecule is a bisphosphonate.

In some embodiments of the invention composition, optionally in combination with any of the various embodiments disclosed herein, the carrier is a non-aqueous carrier.

In some embodiments of the invention composition, optionally in combination with any of the various embodiments disclosed herein, the bone-targeting molecule is one of

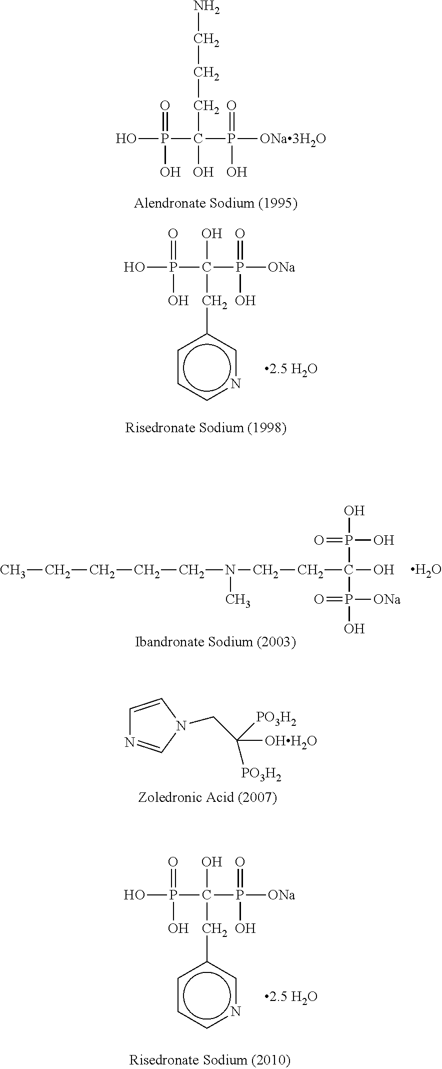

In some embodiments of the invention composition, optionally in combination with any of the various embodiments disclosed herein, the composition is in a formulation for local delivery or systemic delivery.

In some embodiments of the invention composition, optionally in combination with any of the various embodiments disclosed herein, the formulation for local delivery is a topical formulation.

In some embodiments of the invention composition, optionally in combination with any of the various embodiments disclosed herein, the formulation is an injection formulation.

In some embodiments of the invention composition, optionally in combination with any of the various embodiments disclosed herein, the formulation comprises a hydrogel or an emulsion.

In some embodiments of the invention composition, optionally in combination with any of the various embodiments disclosed herein, the formulation comprises hyaluronic acid, provitamin B5, anti-itching agent, and/or vitamin C.

### Emulsion

As used herein, the term “emulsion” refers to an oil-in-water (“O/W”) or water-in-oil (“W/O”) emulsion. Generally, in an emulsion formulation, the retinoic acid useful in the invention composition is dissolved in an oil phase, which emulsifies in the presence of an emulsifier (e.g., emulsifying wax) with a water phase.

Generally, in an emulsion, water soluble ingredients, e.g., glycerin or water soluble vitamins such as vitamin C, B, provitamin B5, or caffeine, are dissolved in the water phase, and oil soluble ingredients such as retinoic acid or cannabidiol (CBD) or vitamin A or vitamin E are dissolved in the oil phase. The oil phase can include a mineral oil or plant oil, e.g., hemp oil, Jojoba oil, almond oil, olive oil, vegetable oil, grape seed oil. The oil phase can also be a mixture of the mineral oil along with one or more of the plant oils.

The oil phase can include any of the oil soluble ingredients in addition to retinoic acid, including CBD, vitamin A or vitamin E. The water phase can include any of the water soluble ingredients herein, e.g., vitamin C, vitamin B, provitamin B5, caffeine, etc., or a combination thereof.

Methods and materials for forming an emulsion is well known in the art.

### Carriers

The present invention involves compositions useful for practicing the therapeutic methods described herein. Therapeutic compositions contain a physiologically tolerable carrier together with an active agent as described herein, dissolved or dispersed therein as an active ingredient. In a preferred embodiment, the therapeutic composition is not immunogenic when administered to a mammal or human patient for therapeutic purposes. As used herein, the terms “pharmaceutically acceptable”, “physiologically tolerable” and grammatical variations thereof, as they refer to compositions, carriers, diluents and reagents, are used interchangeably and represent that the materials are capable of administration to or upon a mammal without the production of undesirable physiological effects such as nausea, dizziness, gastric upset and the like. A pharmaceutically acceptable carrier will not promote the raising of an immune response to an agent with which it is admixed, unless so desired. The preparation of a pharmacological composition that contains active ingredients dissolved or dispersed therein is well understood in the art and need not be limited based on formulation. Typically such compositions are prepared as injectable either as liquid solutions or suspensions, however, solid forms suitable for solution, or suspensions, in liquid prior to use can also be prepared. The preparation can also be emulsified or presented as a liposome composition. The active ingredient can be mixed with excipients which are pharmaceutically acceptable and compatible with the active ingredient and in amounts suitable for use in the therapeutic methods described herein. Suitable excipients include, for example, water, saline, dextrose, glycerol, ethanol or the like and combinations thereof. In addition, if desired, the composition can contain minor amounts of auxiliary substances such as wetting or emulsifying agents, pH buffering agents and the like which enhance the effectiveness of the active ingredient. The therapeutic composition of the present invention can include pharmaceutically acceptable salts of the components therein. Pharmaceutically acceptable salts include the acid addition salts that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like. Physiologically tolerable carriers are well known in the art. Exemplary liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. The amount of an active agent used in the methods described herein that will be effective in the treatment of a particular disorder or condition will depend on the nature of the disorder or condition, and can be determined by standard clinical techniques.

Pharmaceutically acceptable carrier is well known in the art. Examples of such carrier includes, e.g., salient, for liquid or suspension formulations, natural or synthetic polymeric materials for burst or sustained release formulations or targeted delivery formulations. Some examples of the carriers are further described in detail below.

### Polymeric Materials

In some embodiments, the carrier disclosed herein can be a polymeric material Exemplary polymeric material that can be used here include but are not limited to a biocompatible or bioabsorbable polymer that is one or more of poly(DL-lactide), poly(L-lactide), poly(L-lactide), poly(L-lactide-co-DL-lactide), polymandelide, polyglycolide, poly(lactide-co-glycolide), poly(D,L-lactide-co-glycolide), poly(L-lactide-co-glycolide), poly(ester amide), poly(ortho esters), poly(glycolic acid-co-trimethylene carbonate), poly(D,L-lactide-co-trimethylene carbonate), poly(trimethylene carbonate), poly(lactide-co-caprolactone), poly(glycolide-co-caprolactone), poly(tyrosine ester), polyanhydride, derivatives thereof. In some embodiments, the polymeric material comprises a combination of these polymers.

In some embodiments, the polymeric material comprises poly(D,L-lactide-co-glycolide). In some embodiments, the polymeric material comprises poly(D,L-lactide). In some embodiments, the polymeric material comprises poly(L-lactide). [006 Additional exemplary polymers include but are not limited to poly(D-lactide) (PDLA), polymandelide (PM), polyglycolide (PGA), poly(L-lactide-co-D,L-lactide) (PLDLA), poly(D,L-lactide) (PDLLA), poly(D,L-lactide-co-glycolide) (PLGA) and poly(L-lactide-co-glycolide) (PLLGA). With respect to PLLGA, the stent scaffolding can be made from PLLGA with a 25 mole % of GA between 5-15 mol %. The PLLGA can have a mole % of (LA:GA) of 85:15 (or a range of 82: 18 to 88: 12), 95:5 (or a range of 93:7 to 97:3), or commercially available PLLGA products identified as being 85: 15 or 95:5 PLLGA. The examples provided above are not the only polymers that may be used. Many other examples can be provided, such as those found in Polymeric Biomaterials, second edition, edited by Severian Dumitriu; chapter 4.

In some embodiments, polymers that are more flexible or that have a lower modulus that those mentioned above may also be used. Exemplary lower modulus bioabsorbable polymers include, polycaprolactone (PCL), poly(trimethylene carbonate) (PTMC), polydioxanone (PDO), poly(3-hydrobutyrate) (PHB), poly(4-hydroxybutyrate) (P4HB), poly(hydroxyalkanoate) (PHA), and poly(butylene succinate), and blends and copolymers thereof.

In exemplary embodiments, higher modulus polymers such as PLLA or PLLGA may be blended with lower modulus polymers or copolymers with PLLA or PLGA. The blended lower modulus polymers result in a blend that has a higher fracture toughness than the high modulus polymer. Exemplary low modulus copolymers include poly(L-lactide)-b-polycaprolactone (PLLA-b-PCL) or poly(L-lactide)-co-polycaprolactone (PLLA-co-PCL). The composition of a blend can include 1-5 wt % of low modulus polymer.

More exemplary polymers include but are not limited to at least partially alkylated polyethyleneimine (PEI); at least partially alkylated poly(lysine); at least partially alkylated polyornithine; at least partially alkylated poly(amido amine), at least partially alkylated homo- and co-polymers of vinylamine; at least partially alkylated acrylate containing aminogroups, copolymers of vinylamine containing aminogroups with hydrophobic monomers, copolymers of acrylate containing aminogroups with hydrophobic monomers, and amino containing natural and modified polysaccharides, polyacrylates, polymethacryates, polyureas, polyurethanes, polyolefins, polyvinylhalides, polyvinylidenehalides, polyvinylethers, polyvinylaromatics, polyvinylesters, polyacrylonitriles, alkyd resins, polysiloxanes and epoxy resins, and mixtures thereof. [006 Additional examples of biocompatible biodegradable polymers include, without limitation, polycaprolactone, poly(L-lactide), poly(D,L-lactide), poly(D,L-lactide-co-PEG) block copolymers, poly(D,L-lactide-co-trimethylene carbonate), poly(lactide-co-glycolide), polydioxanone (PDS), polyorthoester, polyanhydride, poly(glycolic acid-co-trimethylene carbonate), polyphosphoester, polyphosphoester urethane, poly(amino acids), polycyanoacrylates, poly(trimethylene carbonate), poly(iminocarbonate), polycarbonates, polyurethanes, polyalkylene oxalates, polyphosphazenes, PHA-PEG, and combinations thereof. The PHA may include poly(a-hydroxyacids), poly(P-hydroxyacid) such as poly(3-hydroxybutyrate) (PHB), poly(3-hydroxybutyrate-co-valerate) (PHBV), poly(3-hydroxyproprionate) (PHP), poly(3-hydroxyhexanoate) (PHH), or poly(4-hydroxyacid) such as poly poly(4-hydroxybutyrate), poly(4-hydroxyvalerate), poly(4-hydroxyhexanoate), poly(hydroxyvalerate), poly(tyrosine carbonates), poly(tyrosine arylates), poly(ester amide), polyhydroxyalkanoates (PHA), poly(3-hydroxyalkanoates) such as poly(3-hydroxypropanoate), poly(3-hydroxybutyrate), poly(3-hydroxyvalerate), poly(3-hydroxyhexanoate), poly(3-hydroxyheptanoate) and poly(3-hydroxyoctanoate), poly(4-hydroxyalkanaote) such as poly(4-hydroxybutyrate), poly(4-hydroxyvalerate), poly(4-hydroxyhexanote), poly(4-hydroxyheptanoate), poly(4-hydroxyoctanoate) and copolymers including any of the 3-hydroxyalkanoate or 4-hydroxyalkanoate monomers described herein or blends thereof, poly(D,L-lactide), poly(L-lactide), polyglycolide, poly(D,L-lactide-co-glycolide), poly(L-lactide-co-glycolide), polycaprolactone, poly(lactide-co-caprolactone), poly(glycolide-co-caprolactone), poly(dioxanone), poly(ortho esters), poly(anhydrides), poly(tyrosine carbonates) and derivatives thereof, poly(tyrosine ester) and derivatives thereof, poly(imino carbonates), poly(glycolic acid-co-trimethylene carbonate), polyphosphoester, polyphosphoester urethane, poly(amino acids), polycyanoacrylates, poly(trimethylene carbonate), poly(iminocarbonate), polyphosphazenes, silicones, polyesters, polyolefms, polyisobutylene and ethylene-alphaolefin copolymers, acrylic polymers and copolymers, vinyl halide polymers and copolymers, such as polyvinyl chloride, polyvinyl ethers, such as polyvinyl methyl ether, polyvinylidene halides, such as polyvinylidene chloride, polyacrylonitrile, polyvinyl ketones, polyvinyl aromatics, such as polystyrene, polyvinyl esters, such as polyvinyl acetate, copolymers of vinyl monomers with each other and olefins, such as ethylene-methyl methacrylate copolymers, acrylonitrile-styrene copolymers, ABS resins, and ethylene-vinyl acetate copolymers, polyamides, such as Nylon 66 and polycaprolactam, alkyd resins, polycarbonates, polyoxymethylenes, polyimides, polyethers, poly(glyceryl sebacate), poly(propylene fumarate), poly(n-butyl methacrylate), poly(sec-butyl methacrylate), poly(isobutyl methacrylate), poly(tert-butyl methacrylate), poly(n-propyl methacrylate), poly(isopropyl methacrylate), poly(ethyl methacrylate), poly(methyl methacrylate), epoxy resins, polyurethanes, rayon, rayon-triacetate, cellulose acetate, cellulose butyrate, cellulose acetate butyrate, cellophane, cellulose nitrate, cellulose propionate, cellulose ethers, carboxymethyl cellulose, polyethers such as poly(ethylene glycol) (PEG), copoly(ether-esters) (e.g. poly(ethylene oxide-co-lactic acid) (PEO/PLA)), polyalkylene oxides such as poly(ethylene oxide), poly(propylene oxide), poly(ether ester), polyalkylene oxalates, phosphoryl choline containing polymer, choline, poly(aspirin), polymers and co-polymers of hydroxyl bearing monomers such as 2-hydroxyethyl methacrylate (HEMA), hydroxypropyl methacrylate (HPMA), hydroxypropylmethacrylamide, PEG acrylate (PEGA), PEG methacrylate, methacrylate polymers containing 2-methacryloyloxyethyl-phosphorylcholine (MPC) and n-vinyl pyrrolidone (VP), carboxylic acid bearing monomers such as methacrylic acid (MA), acrylic acid (AA), alkoxymethacrylate, alkoxyacrylate, and 3-trimethylsilylpropyl methacrylate (TMSPMA), poly(styrene-isoprene-styrene)-PEG (SIS-PEG), polystyrene-PEG, polyisobutylene-PEG, polycaprolactone-PEG (PCL-PEG), PLA-PEG, poly(methyl methacrylate), MED610, poly(methyl methacrylate)-PEG (PMMA-PEG), polydimethylsiloxane-co-PEG (PDMS-PEG), poly(vinylidene fluoride)-PEG (PVDF-PEG), PLURONIC™ surfactants (polypropylene oxide-co-polyethylene glycol), poly(tetramethylene glycol), hydroxy functional poly(vinyl pyrrolidone), biomolecules such as collagen, chitosan, alginate, fibrin, fibrinogen, cellulose, starch, dextran, dextrin, hyaluronic acid, fragments and derivatives of hyaluronic acid, heparin, fragments and derivatives of heparin, glycosamino glycan (GAG), GAG derivatives, polysaccharide, elastin, elastin protein mimetics, or combinations thereof.

In some embodiments, polyethylene is used to construct at least a portion of the device. For example, polyethylene can be used in an orthopedic implant on a surface that is designed to contact another implant, as such in a joint or hip replacement. Polyethylene is very durable when it comes into contact with other materials. When a metal implant moves on a polyethylene surface, as it does in most joint replacements, the contact is very smooth and the amount of wear is minimal. Patients who are younger or more active may benefit from polyethylene with even more resistance to wear. This can be accomplished through a process called crosslinking, which creates stronger bonds between the elements that make up the polyethylene. The appropriate amount of crosslinking depends on the type of implant.

For example, the surface of a hip implant may require a different degree of crosslinking than the surface of a knee implant.

Additional examples of polymeric materials can be found, for example, in U.S. Pat. No. 6,127,448 to Domb, US Pat. Pub. No. 2004/0148016 by Klein and Brazil, US Pat. Pub. No. 2009/0169714 by Burghard et al., U.S. Pat. No. 6,406,792 to Briquet et al, US Pat. Pub. No. 2008/0003256 by Martens et al, each of which is hereby incorporated by reference herein in its entirety.

### Methods of Fabrication

In a further aspect of the present invention, it is provided a method of fabricating a composition for relief or management of a pain associated with a disorder, comprising providing a therapeutically effective amount of retinoic acid and a carrier, and dissolving or dispersing the therapeutically effective amount of retinoic acid in the carrier to form the composition.

In some embodiments of the invention method, optionally in combination with any of the various embodiments disclosed herein, the disorder is of an organ, muscle, joint, or a skeletal structure.

In some embodiments of the invention method, optionally in combination with any of the various embodiments disclosed herein, the disorder is of an acute condition or a chronic condition.

In some embodiments of the invention method, optionally in combination with any of the various embodiments disclosed herein, the retinoic acid comprising 0.01% to 2% of the total weight of the composition.

In some embodiments of the invention method, optionally in combination with any of the various embodiments disclosed herein, the composition further comprises a bone-targeting molecule for directing the retinoic acid to a skeletal tissue.

In some embodiments of the invention method, optionally in combination with any of the various embodiments disclosed herein, the bone-targeting molecule is a bisphosphonate.

In some embodiments of the invention method, optionally in combination with any of the various embodiments disclosed herein, the carrier is a non-aqueous carrier.

In some embodiments of the invention method, optionally in combination with any of the various embodiments disclosed herein, the bone-targeting molecule is one of

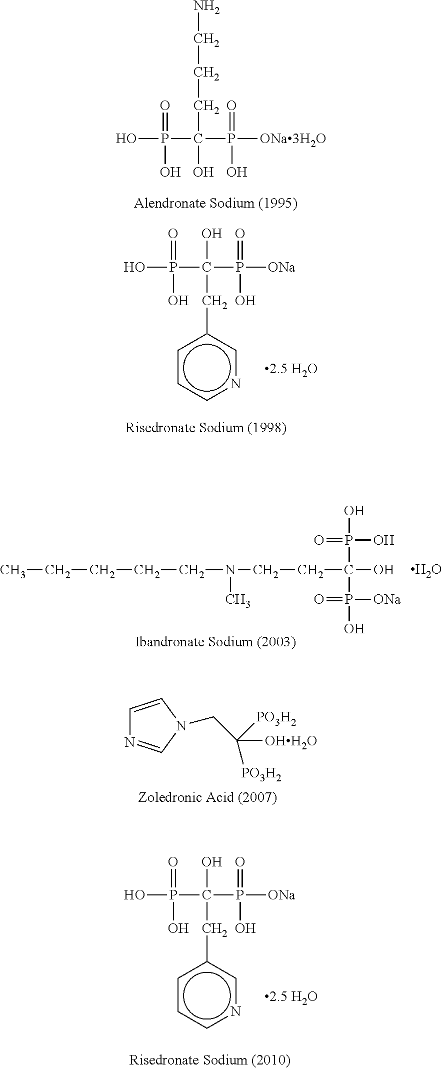

In some embodiments of the invention method, optionally in combination with any of the various embodiments disclosed herein, the composition is in a formulation for local delivery or systemic delivery.

In some embodiments of the invention method, optionally in combination with any of the various embodiments disclosed herein, the formulation for local delivery is a topical formulation.

In some embodiments of the invention method, optionally in combination with any of the various embodiments disclosed herein, the formulation is an injection formulation.

In some embodiments of the invention method, optionally in combination with any of the various embodiments disclosed herein, the formulation comprises a hydrogel or an emulsion.

In some embodiments of the invention method, optionally in combination with any of the various embodiments disclosed herein, the formulation comprises hyaluronic acid, provitamin B5, anti-itching agent, and/or vitamin C.

### Methods of Use

In another aspect of the present invention, it is provided a method for relief or management of a pain associated with a disorder, comprising administering to a subject in need thereof a composition that comprises a therapeutically effective amount of retinoic acid dissolved or dispersed in a carrier to a site having the pain or disorder, causing a recognizable reduction of pain in the subject.

In some embodiments of the invention method, optionally in combination with any of the various embodiments disclosed herein, the disorder is of an organ, muscle, joint, or a skeletal structure.

In some embodiments of the invention method, optionally in combination with any of the various embodiments disclosed herein, the disorder is of an acute condition or a chronic condition.

In some embodiments of the invention method, optionally in combination with any of the various embodiments disclosed herein, the retinoic acid comprising 0.01% to 2% of the total weight of the composition.

In some embodiments of the invention method, optionally in combination with any of the various embodiments disclosed herein, the composition further comprises a bone-targeting molecule for directing the retinoic acid to a skeletal tissue.

In some embodiments of the invention method, optionally in combination with any of the various embodiments disclosed herein, the bone-targeting molecule is a bisphosphonate.

In some embodiments of the invention method, optionally in combination with any of the various embodiments disclosed herein, the carrier is a non-aqueous carrier.

In some embodiments of the invention method, optionally in combination with any of the various embodiments disclosed herein, the bone-targeting molecule is one of

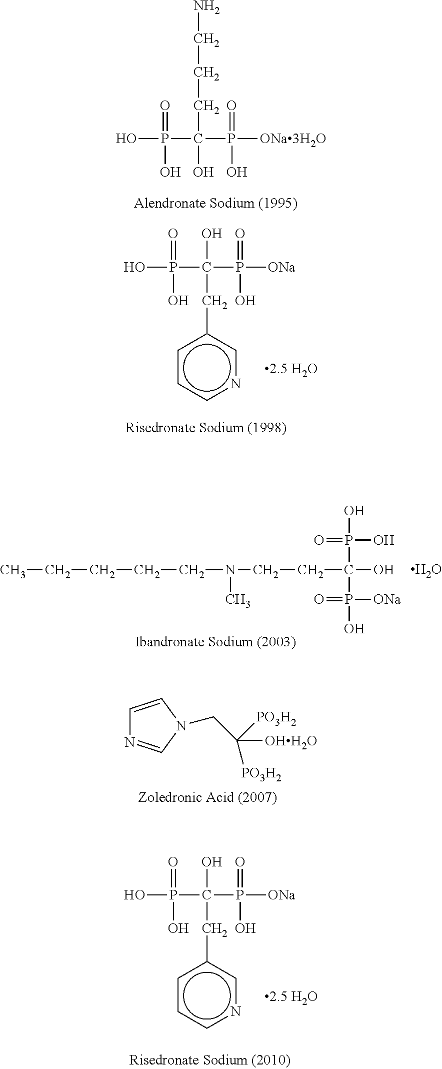

In some embodiments of the invention method, optionally in combination with any of the various embodiments disclosed herein, the composition is in a formulation for local delivery or systemic delivery.

In some embodiments of the invention method, optionally in combination with any of the various embodiments disclosed herein, the formulation for local delivery is a topical formulation.

In some embodiments of the invention method, optionally in combination with any of the various embodiments disclosed herein, the formulation is an injection formulation.

In some embodiments of the invention method, optionally in combination with any of the various embodiments disclosed herein, the formulation comprises a hydrogel or an emulsion.

In some embodiments of the invention method, optionally in combination with any of the various embodiments disclosed herein, the formulation comprises hyaluronic acid, provitamin B5, anti-itching agent, and/or vitamin C.

In some embodiments of the invention method, optionally in combination with any of the various embodiments disclosed herein, the subject is a patient, a pet or horse.

In a further aspect of the present invention, it is provided a method of using a composition for relief or management of a pain associated with a disorder in a subject in need thereof, the composition comprising a therapeutically effective amount of retinoic acid dissolved or dispersed in a carrier, wherein application of the composition to a site having the pain or disorder causes a recognizable reduction of pain in the subject.

In some embodiments of the invention method, optionally in combination with any of the various embodiments disclosed herein, the disorder is of an organ, muscle, joint, or a skeletal structure.

In some embodiments of the invention method, optionally in combination with any of the various embodiments disclosed herein, the disorder is of an acute condition or a chronic condition.

In some embodiments of the invention method, optionally in combination with any of the various embodiments disclosed herein, the retinoic acid comprising 0.01% to 2% of the total weight of the composition.

In some embodiments of the invention method, optionally in combination with any of the various embodiments disclosed herein, the composition further comprises a bone-targeting molecule for directing the retinoic acid to a skeletal tissue.

In some embodiments of the invention method, optionally in combination with any of the various embodiments disclosed herein, the bone-targeting molecule is a bisphosphonate.

In some embodiments of the invention method, optionally in combination with any of the various embodiments disclosed herein, the carrier is a non-aqueous carrier.

In some embodiments of the invention method, optionally in combination with any of the various embodiments disclosed herein, the bone-targeting molecule is one of

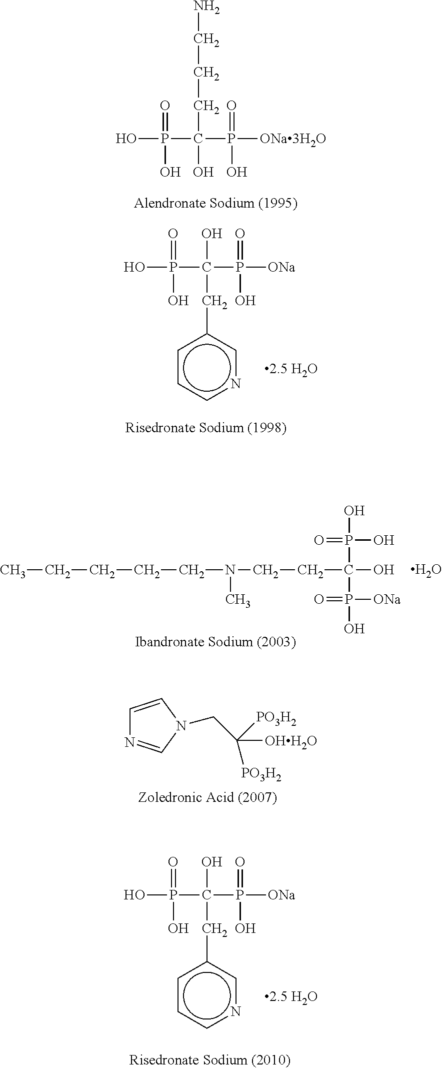

In some embodiments of the invention method, optionally in combination with any of the various embodiments disclosed herein, the composition is in a formulation for local delivery or systemic delivery.

In some embodiments of the invention method, optionally in combination with any of the various embodiments disclosed herein, the formulation for local delivery is a topical formulation.

In some embodiments of the invention method, optionally in combination with any of the various embodiments disclosed herein, the formulation is an injection formulation.

In some embodiments of the invention method, optionally in combination with any of the various embodiments disclosed herein, the formulation comprises a hydrogel or an emulsion.

In some embodiments of the invention method, optionally in combination with any of the various embodiments disclosed herein, the formulation comprises hyaluronic acid, provitamin B5, anti-itching agent, and/or vitamin C.

In some embodiments of the invention method, optionally in combination with any of the various embodiments disclosed herein, the subject is a patient, a pet or horse.

### Dosage and Administration

The dosage can be determined by one of skill in the art and can also be adjusted by the individual physician in the event of any complication. Typically, the dosage ranges from 0.0005 mg/kg body weight to 1 g/kg body weight. In some embodiments, the dosage range is from 0.001 mg/kg body weight to 0.5 g/kg body weight, from 0.0005 mg/kg body weight to 0.1 g/kg body weight, from 0.001 mg/kg body weight to 0.05 g/kg body weight.

As another alternative, dosage is selected for localized delivery and is not necessary selected to body weight or to achieve a certain serum level, but to achieve a localized effect, e.g., as for a localized injection, implantation or other localized administration to the eye. Administration of the doses recited above can be repeated for a limited period of time. In some embodiments, the doses are given once a day, or multiple times a day, for example but not limited to three times a day. In a preferred embodiment, the doses recited above are administered daily for several weeks or months. The duration of treatment depends upon the subject's clinical progress and responsiveness to therapy. Continuous, relatively low maintenance doses are contemplated after an initial higher therapeutic dose.

Agents useful in the methods and compositions described herein can be administered topically, intravenously (by bolus or continuous infusion), orally, by inhalation, intraperitoneally, intramuscularly, subcutaneously, intracavity, and can be delivered by peristaltic means, if desired, or by other means known by those skilled in the art. It is preferred that the agents for the methods described herein are administered topically to the eye. For the treatment of tumors, the agent can be administered systemically, or alternatively, can be administered directly to the tumor e.g., by intratumor injection or by injection into the tumor's primary blood supply.

Therapeutic compositions containing at least one agent disclosed herein can be conventionally administered in a unit dose. The term “unit dose” when used in reference to a therapeutic composition refers to physically discrete units suitable as unitary dosage for the subject, each unit containing a predetermined quantity of active material calculated to produce the desired therapeutic effect in association with the required physiologically acceptable diluent, i.e., carrier, or vehicle.

The compositions are administered in a manner compatible with the dosage formulation, and in a therapeutically effective amount. The quantity to be administered and timing depends on the subject to be treated, capacity of the subject's system to utilize the active ingredient, and degree of therapeutic effect desired. An agent can be targeted by means of a targeting moiety, such as e.g., an antibody or targeted liposome technology. Antibody-based or non-antibody-based targeting moieties can be employed to deliver a ligand or the inhibitor to a target site. Preferably, a natural binding agent for an unregulated or disease associated antigen is used for this purpose.

Precise amounts of active ingredient required to be administered depend on the judgment of the practitioner and are particular to each individual. However, suitable dosage ranges for systemic application are disclosed herein and depend on the route of administration. Suitable regimes for administration are also variable, but are typified by an initial administration followed by repeated doses at one or more intervals by a subsequent injection or other administration. Alternatively, continuous intravenous infusion sufficient to maintain concentrations in the blood in the ranges specified for in vivo therapies are contemplated.

An agent may be adapted for catheter-based delivery systems including coated balloons, slow-release drug-eluting stents or other drug-eluting formats, microencapsulated PEG liposomes, or nanobeads for delivery using direct mechanical intervention with or without adjunctive techniques such as ultrasound.

It is understood that the foregoing detailed description and the following examples are illustrative only and are not to be taken as limitations upon the scope of the invention.

Various changes and modifications to the disclosed embodiments, which will be apparent to those of skill in the art, may be made without departing from the spirit and scope of the present invention. Further, all patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents are based on the information available to the applicants and do not constitute any admission as to the correctness of the dates or contents of these documents. The following examples illustrate rather than limit the embodiments of the present invention.

Those skilled in the art will know, or be able to ascertain, using no more than routine experimentation, many equivalents to the specific embodiments of the invention described herein. These and all other equivalents are intended to be encompassed by the following claims.

### EXAMPLES

The examples below further illustrate, and shall not be construed to limit, the scope of present invention.

### Examples 1-5. Topical Formulation for Skin

An emulsion composition of the present invention comprising 0.5% RA with 25% hemp oil was formed and used on subjects suffering from joint pain and back pain. The test use information is summarized below:

| | | Condition | Condition | |
|---|---|---|---|---|
| Example | Gender/Age | before use | after use | Use Length |
| 1 | Male/72 | OA, joint pain, | 90% pain | 3 weeks |
| | | unable to walk | disappeared | |
| 2 | Male/56 | OA, middle left | Pain | 2 weeks |
| | | finger joint pain | disappeared | |
| 3 | Female/62 | RA, hip | 90% pain on | 2 weeks |
| | | replacement, | knee, hip | |
| | | knee joint pain | and back | |
| | | | disappeared | |
| 4 | Female/50 | OA, left hand | Pain | 2 weeks |
| | | wrist swelling | disappeared | |
| | | and pain | | |
| 5 | Male/64 | OA, knee injury | Pain | 2 weeks |
| | | with swelling | disappeared | |

It is understood that the foregoing detailed description and the following examples are illustrative only and are not to be taken as limitations upon the scope of the invention. Various changes and modifications to the disclosed embodiments, which will be apparent to those of skill in the art, may be made without departing from the spirit and scope of the present invention. Further, all patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents are based on the information available to the applicants and do not constitute any admission as to the correctness of the dates or contents of these documents.

The following examples illustrate rather than limit the embodiments of the present invention.

Those skilled in the art will know, or be able to ascertain, using no more than routine experimentation, many equivalents to the specific embodiments of the invention described herein. These and all other equivalents are intended to be encompassed by the following claims.

1. A composition for relief or management of a pain associated with a disorder which is osteoarthritis or rheumatoid arthritis in a subject in need thereof, comprising a therapeutically effective amount of retinoic acid dissolved or dispersed in a carrier, wherein application of the composition to a site having the disorder causes a recognizable reduction of pain in the subject.

2. The composition according to claim 1, wherein the disorder is of joint, or a skeletal structure.

3. The composition according to claim 1, wherein the disorder is of an acute condition or a chronic condition.

4. The composition according to claim 1, wherein the retinoic acid comprising 0.01% to 2% of the total weight of the composition.

5. The composition of claim 1, wherein the composition further comprises a bone-targeting molecule for directing the retinoic acid to a skeletal tissue.

6. The composition of claim 5, wherein the bone-targeting molecule is a bisphosphonate.

7. The composition of claim 1, wherein the carrier is a non-aqueous carrier.

8. The composition of claim 5, wherein the bone-targeting molecule is one of

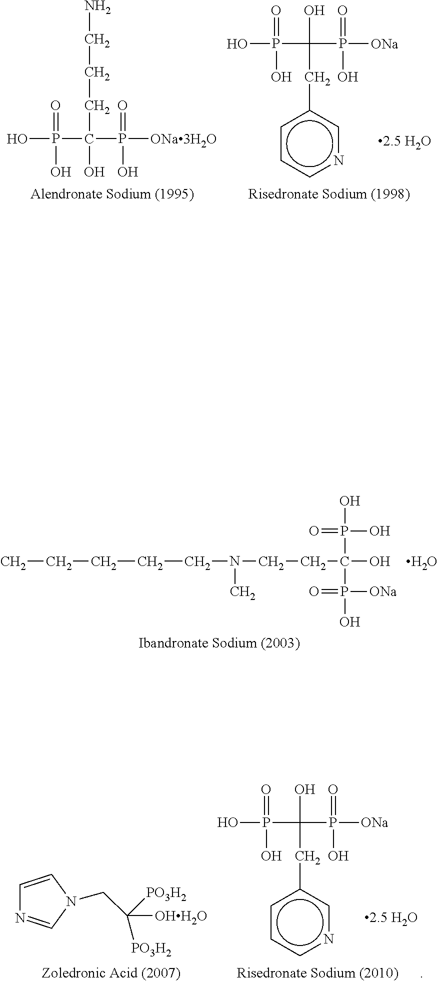

9. The composition of claim 1, which is in a formulation for local delivery or systemic delivery.

10. The composition of claim 9, wherein the formulation for local delivery is a topical formulation.

11. The composition of claim 9, wherein the formulation is an injection formulation.

12. The composition of claim 9, wherein the formulation comprises a hydrogel or an emulsion.

13. The composition of claim 9, wherein the formulation comprises hyaluronic acid, provitamin B5, anti-itching agent, and/or vitamin C.

14. A method for relief or management of a pain associated with a disorder which is osteoarthritis or rheumatoid arthritis, comprising administering to a subject in need thereof a composition that comprises a therapeutically effective amount of retinoic acid dissolved or dispersed in a carrier to a site having the disorder, causing a recognizable reduction of pain in the subject.

15. The method of claim 14, wherein the composition is according to claim 2.

16. The method of claim 14, wherein the subject is a patient, a pet or horse.

17. (canceled)

18. (canceled)

19. A method of fabricating a composition for relief or management of a pain associated with a disorder which is osteoarthritis or rheumatoid arthritis, comprising providing a therapeutically effective amount of retinoic acid and a carrier, and dissolving or dispersing the therapeutically effective amount of retinoic acid in the carrier to form the composition.

20. The method according to claim 19, wherein the composition is according to 2.
